(19) United States
(12) Patent Application Publication
(10) Pub. No.: US 2026/0309704 A1
(43) Pub. Date: Oct. 8, 2026

(54) System and Method for Single Protein Detection by Digital Plasmonic Nanobubbles

(71) Applicant: Board of Regents, The University of Texas System, Austin, TX (US)

(72) Inventors: Zhenpeng Qin, Allen, TX (US); Tingting Zhang, Dallas (KR); Ye Gao, Plano, TX (US)

(21) Appl. No.: 19/460,484

(22) Filed: Jan. 27, 2026

Related U.S. Application Data
(60) Provisional application No. 63/750,243, filed on Jan. 27, 2025.

(51) Int. Cl. G01N 33/543; B82Y 15/00; G01N 21/64; G01N 33/551; G16B 40/10

(52) U.S. Cl. CPC G01N 33/54346; G01N 21/6402; G01N 33/551; G16B 40/10; B82Y 15/00; G01N 2333/135

(57) ABSTRACT

Disclosed are systems, methods, and kits for enzyme-free, compartment-free digital detection of target analytes, such as single protein biomarkers and multivalent targets, using transient plasmonic nanobubbles (PNBs) generated around gold nanoparticles. In a bead-based embodiment, capture antibodies on microbeads bind the analyte, followed by labeling with detection antibody-conjugated gold nanoparticles. After washing, labeled complexes are flowed through a detection zone and exposed to pulsed laser light, generating individual PNBs detected as binary scattering signals via a probe laser and photodetector. Poisson statistics enable absolute quantification at attomolar sensitivity. In a homogeneous embodiment, pairs of antibody-conjugated nanoparticles aggregate upon binding multivalent targets, producing distinguishable PNB signals for rapid, wash-free detection. The platform offers high throughput, minimal sample volume (<10 μL), room-temperature operation, and simplified instrumentation for ultrasensitive biomarker and viral diagnostics.

### RELATED APPLICATIONS

This Application claims priority to U.S. Provisional Patent Application Ser. No. 63/750,243 filed Jan. 27, 2025 which is incorporated herein by reference in its entirety.

### STATEMENT REGARDING FEDERALLY FUNDED RESEARCH

This invention was made with government support under R01AI151374 awarded by the National Institutes of Health. The government has certain rights in the invention.

### FIELD OF INVENTION

This application is generally directed to the field of immunoassays, more specifically ultrasensitive analyte detection.

### BACKGROUND

Protein biomarkers are essential for diagnosing and monitoring diseases such as cancer, neurodegenerative disorders, infectious diseases, and cardiovascular conditions, particularly when early intervention requires detection at ultra-low (attomolar or femtomolar) concentrations.

Immunoassays can be broadly classified into two categories: analog immunoassays and digital immunoassays.

Analog immunoassays produce a continuous signal whose intensity is directly proportional to the concentration of the target analyte in the sample. Common examples include enzyme-linked immunosorbent assay (ELISA), mass spectrometry-based assays, and Western blotting. These methods are widely used due to their simplicity and established protocols but are fundamentally limited in sensitivity. At very low analyte concentrations, the signal approaches the background noise level, resulting in poor signal-to-noise ratios and limits of detection typically in the picomolar to nanomolar range.

Digital immunoassays overcome this limitation by converting the detection problem into a binary (present/absent) readout at the single-molecule level. Individual target molecules are isolated into tiny compartments (femtoliter to picoliter volumes, such as microwells or droplets), and the presence of a single molecule in a compartment is detected after amplification, yielding an “on” or “off” signal for each compartment. The fraction of “on” compartments is then analyzed to determine absolute analyte concentration with greatly improved sensitivity. Commercial digital ELISA (dELISA) systems, such as Quanterix SiMOA, achieve attomolar sensitivity and true single-molecule quantification. However, these systems require (1) physical compartmentalization using specialized instrumentation, (2) lengthy enzymatic amplification steps to generate detectable fluorescent signals, and (3) complex, multi-step workflows. These requirements result in high instrument and reagent costs, prolonged assay times (often several hours), and limited compatibility with simple, low-cost, point-of-care settings.

More recent approaches, such as bead-based digital assays using rolling circle amplification (RCA), eliminate the need for physical compartments but still rely on enzymatic amplification, retaining the associated time, reagent, and variability burdens.

Plasmonic nanoparticle-based digital assays avoid enzymatic amplification by using direct optical readouts (scattering or interference) from nanoparticles. However, these methods typically employ imaging-based detection, which restricts throughput, and/or rely on planar surface capture, which suffers from slow diffusion and poor binding efficiency at ultra-low analyte concentrations.

Thus, despite advances in sensitivity, current ultrasensitive immunoassay technologies share common critical limitations: dependence on enzymatic or chemical amplification (introducing time, cost, and variability), requirement for physical compartmentalization and specialized equipment, restricted throughput due to imaging or low-volume processing, and incompatibility with rapid, low-cost, point-of-care workflows that use minimal sample volumes and operate at room temperature.

There remains an unmet need for a simple, rapid, enzyme-free, and compartment-free digital immunoassay platform capable of single-molecule or near-single-molecule sensitivity, high throughput, minimal sample volume, and straightforward instrumentation suitable for both laboratory and point-of-care applications. The present invention addresses this need by providing systems and methods that achieve ultrasensitive digital detection through transient plasmonic nanobubbles generated around plasmonic nanoparticles, enabling instantaneous physical amplification, binary digital counting, and absolute quantification via Poisson statistics without enzymatic amplification or physical compartmentalization.

### SUMMARY

The present invention relates generally to systems, methods, and kits for ultrasensitive digital detection of target analytes in a sample, such as protein biomarkers or multivalent targets (e.g., intact viruses, viral capsids, protein complexes, or multimeric proteins), without requiring enzymatic amplification or physical compartmentalization. The invention leverages the generation of transient plasmonic nanobubbles (PNBs) around plasmonic nanoparticles, which produce detectable scattering signals upon exposure to pulsed laser light. These signals enable binary (“on/off”) digital counting and absolute quantification using Poisson statistics, offering advantages in sensitivity (e.g., attomolar levels for proteins or sub-100 PFU/mL for viruses), speed, simplicity, high throughput, minimal sample volume (e.g., less than 10 μL), room-temperature operation, and compatibility with low-cost, point-of-care workflows.

In one aspect, the invention provides a method for digitally detecting a target analyte in a sample without enzymatic amplification or physical compartmentalization. The method generally involves capturing the target analyte on a solid support using a binding agent to form a captured target analyte, labeling the captured target analyte with a plasmonic nanoparticle to form a labeled solid support, exposing the labeled solid support to pulsed laser light to generate plasmonic nanobubbles around the plasmonic nanoparticles, detecting scattering signals from the plasmonic nanobubbles, and digitally quantifying the target analyte based on the detected scattering signals. In certain embodiments, the target analyte is a protein biomarker, such as prostate-specific antigen (PSA), SARS-CoV-2 spike protein, or respiratory syncytial virus fusion protein (RSV-F). The solid support may be a microbead, such as a silica microbead having a diameter of about 500 nm. The plasmonic nanoparticle may be a gold nanoparticle having a diameter of at least 55 nm or about 65 nm. The binding agent may be an antibody, and the capturing and labeling steps may form a sandwich immunocomplex comprising a capture antibody on the solid support, the target analyte, and a detection antibody conjugated to the plasmonic nanoparticle. The method may further include removing unbound plasmonic nanoparticles after labeling and before exposing to pulsed laser light, such as by washing via filtration or centrifugation. The labeled solid supports may be flowed through a detection zone during exposure to pulsed laser light and detection, where the detection zone is in a fluidic channel or microcapillary. The pulsed laser light may have a wavelength of about 532 nm. Digitally quantifying may comprise counting binary (on/off) scattering signals and applying Poisson statistics to determine absolute concentration. The method may achieve attomolar sensitivity, such as about 32 aM for PSA, about 123 aM for SARS-CoV-2 spike protein, or about 65 aM for RSV-F. The method may be performed at room temperature, and the sample volume may be less than 10 μL.

In another aspect, the invention provides a method for digitally detecting a multivalent target analyte in a sample without enzymatic amplification or physical compartmentalization. The method generally involves mixing the sample with a plurality of pairs of plasmonic nanoparticles, where each member of a pair is conjugated to a different binding agent that recognizes a different epitope on the multivalent target analyte, and where binding of the multivalent target analyte to both binding agents of a pair induces aggregation of the plasmonic nanoparticles; exposing the mixture to pulsed laser light sufficient to generate plasmonic nanobubbles preferentially around aggregated plasmonic nanoparticles compared to non-aggregated plasmonic nanoparticles; detecting transient scattering signals from the plasmonic nanobubbles using a continuous-wave probe laser and a photodetector; and digitally quantifying the target analyte by counting scattering events that satisfy a threshold criterion indicative of aggregation. The method distinguishes aggregated from non-aggregated plasmonic nanoparticles based on at least one of: scattering signal amplitude, scattering signal duration, nanobubble generation threshold fluence, or nanobubble lifetime. In certain embodiments, the plasmonic nanoparticles are gold nanoparticles having a diameter of about 40 nm to about 70 nm or about 50 nm to about 65 nm. The multivalent target analyte may be an intact virus particle (e.g., a respiratory syncytial virus (RSV) particle), a viral capsid, a protein complex, or a multimeric protein. The method may be performed as a wash-free, homogeneous assay. The mixture may be flowed through a microfluidic channel or microcapillary during exposure to the pulsed laser light and detection of scattering signals. The pulsed laser light may have a wavelength of about 520 nm to about 550 nm and a pulse duration of less than about 10 nanoseconds. The continuous-wave probe laser may have a wavelength of about 600 nm to about 650 nm. The threshold criterion for counting a scattering event as indicative of aggregation may comprise at least one of: a scattering signal amplitude at least 2-fold greater than the mean amplitude of signals from non-aggregated nanoparticles; a nanobubble lifetime at least 1.5-fold longer than the mean lifetime from non-aggregated nanoparticles; or a nanobubble generation fluence threshold at least 20% lower than the threshold for non-aggregated nanoparticles. The digital quantification may be performed using Poisson statistics applied to the fraction of detected scattering events that satisfy the aggregation threshold criterion. The method may achieve detection of the multivalent target analyte at a concentration of 100 PFU/mL or lower, such as about 100 PFU/mL or lower for intact RSV. The entire assay may be completed in less than 30 minutes from sample addition to final quantification, and the sample volume used may be less than 10 μL.

In a further aspect, the invention provides a system for digitally detecting a target analyte without enzymatic amplification or physical compartmentalization. The system generally includes solid supports configured to capture the target analyte, plasmonic nanoparticles configured to label the captured target analyte, a pulsed laser configured to generate plasmonic nanobubbles around the plasmonic nanoparticles, a detector configured to detect scattering signals from the plasmonic nanobubbles, and a processor configured to digitally quantify the target analyte based on the scattering signals. In certain embodiments, the solid supports are silica microbeads coated with capture antibodies, and the plasmonic nanoparticles are gold nanoparticles conjugated to detection antibodies. The system may further include a fluidic channel for flowing labeled supports through a laser interrogation zone. The detector may be a photodetector, and the system may further include a probe laser for illuminating the nanobubbles to enhance scattering detection. The processor may be configured to apply Poisson statistics for absolute quantification.

In yet another aspect, the invention provides a system for digitally detecting a target analyte without enzymatic amplification or physical compartmentalization. The system generally includes antibody-conjugated plasmonic nanoparticles configured to aggregate upon binding to the target analyte, a pulsed laser configured to generate plasmonic nanobubbles around the aggregated plasmonic nanoparticles, a detector configured to detect scattering signals from the plasmonic nanobubbles, and a processor configured to digitally quantify the target analyte based on the scattering signals distinguishing aggregated from non-aggregated plasmonic nanoparticles.

In a still further aspect, the invention provides a kit for digitally detecting a target analyte. The kit generally includes solid supports functionalized with binding agents for capturing the target analyte, plasmonic nanoparticles functionalized with binding agents for labeling the captured target analyte, and instructions for exposing labeled supports to pulsed laser light to generate and detect plasmonic nanobubbles for digital quantification without enzymatic amplification or physical compartmentalization. In certain embodiments, the solid supports are silica microbeads, and the plasmonic nanoparticles are gold nanoparticles. The kit may further include buffers for incubation and washing.

In an additional aspect, the invention provides a kit for digitally detecting a target analyte. The kit generally includes pairs of antibody-conjugated plasmonic nanoparticles configured to aggregate upon binding to the target analyte, and instructions for exposing a mixture of the plasmonic nanoparticles and sample to pulsed laser light to generate and detect plasmonic nanobubbles for digital quantification.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method, system, or kit of the invention, and vice versa. Furthermore, systems and kits of the invention can be used to achieve methods of the invention.

### DEFINITIONS

Unless otherwise defined, all technical and scientific terms used herein have the same meaning as commonly understood by one of ordinary skill in the art to which this invention belongs. The following terms, as used in this specification and the appended claims, are defined as follows:

Target analyte or analyte means any molecule, biomolecule, or particle to be detected, including proteins, polypeptides, peptides, protein biomarkers, multivalent targets (e.g., intact virus particles, viral capsids, protein complexes, or multimeric proteins), or other structures presenting multiple epitopes.

Plasmonic nanoparticle or plasmonic nanolabel means a nanoparticle that supports localized surface plasmon resonance and converts incident light energy into localized heat upon suitable laser excitation. Preferred examples include spherical gold nanoparticles (AuNPs), silver nanoparticles, or alloys thereof.

Plasmonic nanobubble or PNB means a transient vapor nanobubble formed around a plasmonic nanoparticle due to rapid localized heating and phase transition of the surrounding medium upon pulsed laser irradiation, producing a detectable, short-lived scattering signal.

Digital detection or digital counting means a binary detection method in which individual binding events or labeled complexes produce discrete “on” or “off” signals, enabling absolute quantification (e.g., via Poisson statistics) without analog intensity measurement.

Solid support or microbead means a particulate substrate for immobilizing capture binding agents in sandwich immunoassays. Preferred examples are silica microbeads or magnetic microbeads, typically about 0.5 to 5 micrometers in diameter.

Magnetic microbead means a microbead incorporating magnetic material, such as iron oxide or other ferromagnetic or superparamagnetic components, that enables magnetic manipulation, including attraction, separation, or isolation using an external magnetic field, while serving as a particulate substrate for immobilizing capture binding agents in sandwich immunoassays.

Binding agent means any moiety that specifically recognizes and binds a target analyte or epitope thereof. Preferred examples include antibodies, antibody fragments, aptamers, lectins, receptors, ligands, or nucleic acid probes.

Sandwich immunocomplex means a ternary complex comprising a capture binding agent immobilized on a solid support, the target analyte, and a detection binding agent conjugated to a plasmonic nanoparticle.

Aggregation means clustering of plasmonic nanoparticles induced by a multivalent target analyte binding to pairs of nanoparticles conjugated to binding agents that recognize distinct epitopes on the analyte.

Wash-free or homogeneous means an assay performed without physical separation or removal of unbound reagents before detection.

Attomolar sensitivity means detection and/or quantification at concentrations in the attomolar range (10⁻¹⁸ M) or lower, corresponding to single-molecule or near-single-molecule resolution.

Poisson statistics means statistical analysis based on the Poisson distribution applied to discrete, rare events to determine absolute analyte concentration from the fraction of positive detection events.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a composition and/or method that “comprises” a list of elements (e.g., components or features or steps) is not necessarily limited to only those elements (or components or features or steps) but may include other elements (or components or features or steps) not expressly listed or inherent to the composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1B. Workflow of the sample preparation for BEACON detection. (A) Capture antibody-modified silica beads are first incubated with target protein in solution, followed by a washing step through a membrane to remove unbound proteins. Detection antibody-functionalized AuNPs are then added to form bead-protein-AuNP sandwich complexes. A second washing step removes excess AuNPs and residual reagents for subsequent digital counting and analysis. (B) Schematic of the BEACON flow detection system. A picosecond pulsed laser (green) excites AuNPs to generate plasmonic nanobubbles, while a co-aligned continuous-wave laser (red) detects the resulting scattering signals. The overlapping beams define a virtual detection volume (~80 pL) within a microcapillary channel (0.2 μm inner diameter), enabling digital single-particle counting. The BEACON system detects target proteins by identifying the presence (‘on’ signal) or absence (‘off’ signal) of AuNP, enabling single-protein resolution and absolute quantification of target proteins.

FIGs. 2A-2E. Single Au nanoprobes detection. (A) Quantitative analysis of Au nanoprobes of different sizes: All nanoparticle sizes were adjusted to a uniform concentration (λ=0.07) using a 2 J/cm² excitation pulse laser. The comparison with theoretical Poisson probability indicates accurate detection of Au nanoprobes with diameters above 55 nm. (B) Quantitative analysis of 65 nm AuNPs: The frequency count (fₒₙ) of detected signals is plotted against the parameter λ to determine the concentrations of 65 nm AuNPs. (C) Correlation analysis between experimental and Poisson statistics: A linear relationship is observed between the experimentally measured frequency count (fₒₙ) and the theoretical Poisson probability, validating the assay's accuracy in quantifying AuNPs concentrations. (D) Quantification of detection probability (fₒₙ) for AuNPs of different sizes (λAuNP=0.1) and pulse energy at 1.7 J/cm2. Data represent mean fₒₙ values from three independent measurements (20,000 pulses each). The dashed line indicates theoretical predictions based on Poisson statistics. AuNPs≥65 nm demonstrate strong agreement with expected Poisson statistics. (E) Calibration curve of fₒₙ versus nanoparticle occupancy λAuNP for 65 nm AuNPs. Inset: linear correlation between experimental fₒₙ and theoretical Poisson statistics.

FIGS. 3A-3E. Avidin detection using the DIAMOND-P assay. (A) Formation of the SiO2-Avidin-Au NP sandwich immune structure: Biotinylated 500 nm SiO₂ beads are initially mixed with avidin for 20 mins at room temperature. Subsequently, biotinylated gold nanoparticles are added with a 15-min incubation period. A filtration washing step follows to remove unbound reagents and excess AuNPs. (B) TEM imaging of post-washing samples with the scale bar of 1 μm, where inset shows the details of single SiO₂-Avidin-AuNP sandwich immune structure. The gold NPs were encircled in yellow. (C) Avidin detection calibration curves: Assays performed with different SiO₂ bead concentrations, corresponding to λ values of 0.07, 0.7, and 7. (D) Data analysis impact on limit of detection: Examines how the number of data points analyzed affects the LOD. (E) Colorimetric analog analysis of post-washing samples.

FIGS. 4A-4D. Prostate-Specific Antigen (PSA) detection with the DIAMOND-P assay. (A) Calibration curve for PSA detection: Calibration is performed using SiO₂ beads at a concentration corresponding to λ=2. The dashed black line represents the limit of detection (LOD), determined to be 31.88 aM. (B) Linear correlation analysis of PSA detection: Demonstrates a direct linear relationship between the frequency count (fₒₙ) measured experimentally and the concentration of PSA.

FIG. 5. RSV samples lysed and analyzed using BEACON with RSV-F-specific antibody pairs. Detection probability (fₒₙ) increased with viral concentration, with a limit of detection of ~6 PFU/mL (~60 genome copies/mL). Data are presented as mean±standard deviation from three independent replicates.

FIG. 6. Example workflow of BEACON/DIAMOND-P.

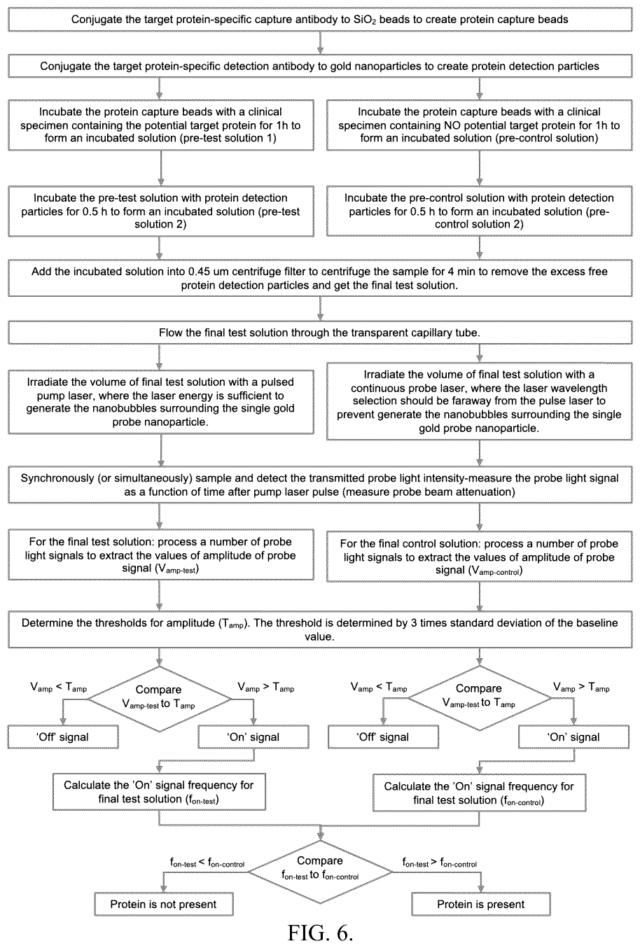

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment and not intended to imply that the scope of the disclosure, including the claims, is limited to that embodiment.

Protein biomarkers and multivalent biological targets (intact viruses, viral capsids, protein complexes, multimeric proteins) are critical for early diagnosis and monitoring of cancer, neurodegenerative diseases, infectious diseases, and cardiovascular conditions. Many clinically relevant targets must be detected at extremely low concentrations-attomolar (10⁻¹⁸ M) for single proteins or sub-100 PFU/mL for intact viruses-to enable early intervention.

Analog immunoassays (ELISA, Western blotting, mass spectrometry) suffer from poor signal-to-noise at low analyte levels, yielding limits of detection (LODs) typically in the picomolar to nanomolar range. Digital immunoassays overcome this by isolating individual binding events and producing binary (present/absent) readouts, enabling absolute quantification via Poisson statistics. Commercial single-molecule platforms (e.g., Quanterix SiMOA) reach attomolar sensitivity but require physical compartmentalization (microwells/droplets), enzymatic amplification, multi-hour workflows, and expensive instrumentation—making them poorly suited to rapid, low-cost, or point-of-care applications.

Enzyme-free and compartment-free digital methods have been explored, yet most remain limited by imaging-based low throughput, slow surface diffusion, or secondary amplification steps. There is therefore a need for a simple, rapid, enzyme-free, compartment-free digital platform capable of attomolar protein detection, sub-100 PFU/mL viral detection, high-throughput flow-based readout, minimal sample volume (<10 μL), room-temperature operation, and compatibility with straightforward optical instrumentation.

### I. Plasmonic Nanobubble-Based Digital Detection

Embodiments described herein use the Digital Plasmonic Nanobubble (DPN) Detection Platform to achieve ultrasensitive digital counting by generating transient plasmonic nanobubbles (PNBs) around plasmonic nanoparticles-most commonly gold nanoparticles (AuNPs)—upon irradiation with short laser pulses. When a plasmonic nanoparticle absorbs energy from a picosecond-duration laser pulse tuned to its localized surface plasmon resonance (typically ~532 nm), it undergoes rapid localized heating that vaporizes the surrounding medium and forms a short-lived vapor nanobubble, with a lifetime typically less than 1 μs. The expansion and collapse of this nanobubble produce a strong, transient increase in light scattering that is detected as a discrete “on” event using a co-aligned continuous-wave probe laser (around 633 nm) and a fast photodetector. In the absence of a suitably sized or energized nanoparticle, no detectable scattering event occurs (“off”). These binary signals enable direct digital counting of individual labeled complexes without enzymatic or chemical amplification, physical compartmentalization into wells or droplets, or wide-field imaging. The platform encompasses two complementary assay formats that share this core physical mechanism but are tailored to different classes of analytes: a heterogeneous bead-based format (BEACON) optimized for monovalent or low-valency protein biomarkers, and a homogeneous aggregation-based format (DIAMOND) optimized for multivalent targets such as intact virus particles.

### A. Bead-Based Heterogeneous Embodiment (Beacon)

The BEACON format (Biomarker Enumeration via Amplified Cavitation Optical Nanobubbles) enables single-protein-level digital quantification using a sandwich immunoassay performed on solid supports. In a typical workflow, 500 nm carboxyl-functionalized silica microbeads are covalently conjugated with capture antibodies using EDC/NHS chemistry and subsequently blocked with BSA. The solid supports may include silica microbeads or magnetic microbeads, typically with diameters ranging from about 0.5 to 5 micrometers, to facilitate efficient capture and handling, such as via magnetic separation for magnetic microbeads. These beads, typically at concentrations ranging from 0.83 to 83 fM, capture the target analyte from the sample during a 20-60 minute incubation at room temperature. After washing via centrifugal filtration to remove unbound analyte, detection antibodies conjugated to approximately 65 nm AuNPs are introduced and incubated for 15-60 minutes to form bead-analyte-AuNP sandwich complexes. Excess unbound nanoparticles are then removed by additional washing steps. The labeled beads are resuspended and flowed through a 200 μm microcapillary, where a 532 nm picosecond pulsed laser excites individual AuNPs-demonstrating optimal performance for nanoparticles 55 nm or larger, with best results at 65 nm—thereby generating plasmonic nanobubbles. A co-aligned 633 nm continuous-wave probe laser illuminates the approximately 80 pL virtual detection volume, and the resulting transient scattering signals are collected through a 532 nm notch filter and recorded by an amplified photodetector. The fraction of laser pulses that produce a detectable scattering event (denoted fₒₙ) is recorded and converted to absolute analyte concentration using Poisson statistics based on the occupancy parameter λ. Experimental validation confirms that single 65 nm AuNP detection closely matches Poisson predictions across a range of concentrations (λ=0.07-7), with reliable counting achieved for AuNPs≥55 nm at fluences of approximately 1.7-2 J/cm². Using avidin as a model analyte on biotinylated beads and AuNPs, BEACON exhibits clear concentration-dependent increases in fₒₙ, strong linear response, and progressive improvement in limit of detection with increasing numbers of detection pulses, reaching a plateau around 40,000-60,000 pulses. For clinically relevant targets, the platform demonstrates limits of detection of approximately 31.88 aM for prostate-specific antigen (PSA) at λ=2 beads with strong linearity in the 0-10 aM range, 123 aM for SARS-CoV-2 spike protein, and 65 aM for respiratory syncytial virus fusion protein (RSV-F). Specificity is confirmed by the RSV-F assay, which produces significantly elevated fₒₙ values compared to background when tested against non-target proteins such as COVID-S and PSA at 160 fM concentrations (more than 1000-fold above the detection limit). When applied to lysed RSV virus samples targeting released RSV-F protein, BEACON achieves a limit of detection of approximately 6 PFU/mL (corresponding to ~60 genome copies/mL), representing a 16-fold improvement over the homogeneous format when evaluated on identical samples. Compared to colorimetric or analog readout of the same complexes, BEACON provides more than 25-fold better sensitivity (18.8 aM versus 551 fM in the avidin model). Thus, BEACON delivers attomolar sensitivity, calibration-free absolute quantification, high specificity, and high-throughput readout (kHz-scale, with potential extension to MHz using faster lasers), all without requiring enzymes or physical partitioning.

### B. Homogeneous Embodiment (Diamond)

The DIAMOND format is designed for rapid, wash-free detection of multivalent analytes. In this approach, the sample is mixed directly with pairs of approximately 40-70 nm AuNPs (optimal range 50-65 nm), where each member of a pair is conjugated to antibodies recognizing distinct epitopes on the target. Binding of a multivalent target induces nanoparticle aggregation or clustering. The mixture is then flowed through the detection channel without any separation or washing steps. Pulsed laser excitation generates plasmonic nanobubbles preferentially around aggregated nanoparticles, producing scattering events with distinguishable characteristics-including higher amplitude, longer lifetime, or lower fluence threshold compared to signals from single or non-aggregated nanoparticles. Scattering events that meet predefined aggregation-specific criteria are counted as positive detection events, and the target concentration is derived via Poisson statistics applied to the fraction of qualifying events. This format achieves detection of intact RSV particles at concentrations of approximately 100 PFU/mL or lower, with total assay times typically under 30 minutes and sample volumes below 10 μL, making it particularly well suited for point-of-care viral diagnostics where speed and minimal hands-on time are critical.

### C. Optical Setup and Readout

Both embodiments employ a compact flow-based optical system consisting of a 532 nm picosecond pulsed pump laser with tunable fluence (typically 0.3-2 J/cm²) for plasmonic nanobubble generation, a co-aligned 600-650 nm continuous-wave probe laser for sensitive scattering detection, fast photodetection electronics with appropriate spectral filtering, and a microfluidic flow cell (such as a 200 μm microcapillary driven by a syringe pump) that defines the interrogation zone. Software handles event counting, application of detection thresholds, and Poisson-based quantification.

### D. Platform Components

The platform of the present invention comprises several components that work synergistically to enable ultrasensitive digital detection of target analytes through the generation and detection of transient plasmonic nanobubbles.

Binding agents are employed to specifically recognize and bind to the target analyte or epitopes thereof. These binding agents broadly encompass any moiety capable of selective interaction with the analyte, including but not limited to antibodies, antibody fragments (such as Fab, Fv, scFv, or diabodies), aptamers, peptides, lectins, receptors, ligands, nucleic acid probes, or engineered binding proteins like affibodies or nanobodies. In specific embodiments, the binding agents are monoclonal or polyclonal antibodies directed against protein biomarkers such as prostate-specific antigen (PSA), SARS-CoV-2 spike protein, or respiratory syncytial virus fusion protein (RSV-F). For detection of multivalent targets, pairs of binding agents are used that recognize distinct epitopes on targets such as intact virus particles, viral capsids, protein complexes, or multimeric proteins, thereby enabling specific capture or aggregation events critical to the claimed detection methods.

Plasmonic nanoparticles serve as labels that support localized surface plasmon resonance and efficiently convert incident light energy into localized heat upon suitable laser excitation. These nanoparticles generally include metallic nanoparticles such as gold, silver, copper, or alloys thereof, in shapes including spheres, rods, shells, or stars, with diameters ranging from about 10 nm to about 200 nm. In preferred embodiments, spherical gold nanoparticles (AuNPs) are utilized, with diameters of at least 55 nm (such as about 65 nm) providing optimal nanobubble generation in heterogeneous bead-based assays, or diameters of about 40 nm to about 70 nm (e.g., 50 nm to 65 nm) in aggregation-based homogeneous assays. The solid supports for heterogeneous assays, such as silica microbeads or magnetic microbeads with diameters of about 0.5 to 5 micrometers, are functionalized with capture binding agents to enable specific analyte binding and subsequent labeling with the plasmonic nanoparticles. The nanoparticles are functionalized with the binding agents through covalent linkages, adsorption, streptavidin-biotin interactions, or other conjugation chemistries, enabling specific labeling of captured analytes or target-induced clustering as described in the claims.

A short-pulsed pump laser is used to irradiate the plasmonic nanoparticles, inducing rapid localized heating and subsequent vapor nanobubble formation via plasmonic resonance. This component encompasses lasers with pulse durations generally less than about 100 nanoseconds (including picosecond or femtosecond pulses) and wavelengths tuned to the plasmon resonance of the nanoparticles, typically in the range of about 400 nm to about 800 nm. In specific implementations, a 532 nm picosecond pulsed laser with tunable fluence from about 0.3 J/cm² to about 2 J/cm² is employed to excite gold nanoparticles in heterogeneous formats, while wavelengths of about 520 nm to about 550 nm are used for aggregated nanoparticle clusters in homogeneous assays, thereby providing the energy necessary to generate detectable plasmonic nanobubbles as recited in the method and system claims.

An optical detection system captures the intense, transient light-scattering signals produced by the expanding and collapsing nanobubbles. This system broadly comprises a light source for illumination and a sensor for signal collection, most commonly a continuous-wave probe laser paired with a photodetector. In particular embodiments, the system includes a probe laser operating at wavelengths of about 600 nm to about 650 nm (such as 633 nm) that is co-aligned with the pump laser, along with spectral filters (e.g., a 532 nm notch filter) and a fast amplified photodetector or avalanche photodiode capable of microsecond-resolution detection of scattering events. This configuration enables binary (“on/off”) event counting of individual labeled complexes or aggregated nanoparticles, supporting the high-throughput, flow-based readout and Poisson statistics-based absolute quantification described throughout the claims.

These four components-binding agents, plasmonic nanoparticles, short-pulsed pump laser, and optical detection system-collectively produce discrete, binary scattering events from plasmonic nanobubbles without requiring enzymatic amplification, chemical signal enhancement, or physical compartmentalization into microwells, droplets, or other discrete volumes. The resulting digital signals enable direct counting of individual binding or aggregation events and absolute quantification of the target analyte using Poisson statistics applied to the fraction of positive detection events, thereby supporting the enzyme-free, compartment-free digital detection methods, systems, and kits claimed herein.

### II. Kits

In certain aspects, the present invention provides kits for performing the digital detection methods using the Digital Plasmonic Nanobubble (DPN) Detection Platform.

In one embodiment, a kit for detecting a target analyte (e.g., protein biomarker) comprises: (a) solid supports functionalized with capture binding agents, e.g., the solid supports may comprise silica microbeads or magnetic microbeads having diameters of about 0.5 to 5 micrometers; (b) plasmonic nanoparticles functionalized with detection binding agents; (c) buffers for incubation and washing; and (d) instructions for capturing the target analyte, labeling with nanoparticles, removing unbound nanoparticles, flowing the complexes through a detection zone, generating and detecting plasmonic nanobubbles, and digitally quantifying the analyte using Poisson statistics.

In another embodiment, a kit for detecting a multivalent target analyte (e.g., intact virus) comprises: (a) pairs of plasmonic nanoparticles (preferably gold nanoparticles of about 40-70 nm), each pair conjugated to different binding agents recognizing different epitopes to induce aggregation upon target binding; (b) buffers for mixing; and (c) instructions for performing a wash-free homogeneous assay, including mixing, flowing, generating and detecting plasmonic nanobubbles preferentially around aggregates, and digitally quantifying the analyte based on aggregation-specific scattering criteria and Poisson statistics.

Kits may further include standards, negative controls, filtration devices, microfluidic components, or instructions for optimizing assay conditions to achieve attomolar (bead-based) or sub-100 PFU/mL (homogeneous) sensitivity. The kits support room-temperature operation, use sample volumes<10 μL, and require no enzymatic amplification or physical compartmentalization, making them suitable for research, clinical, and point-of-care applications.

### III. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### A. Example 1—Beacon

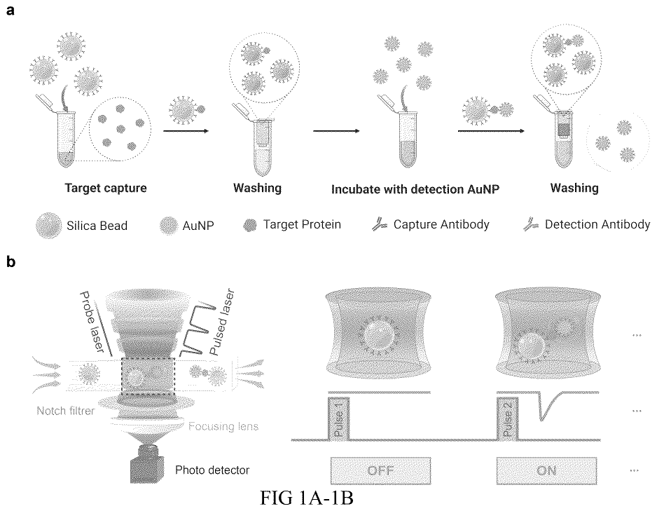

To address the need for ultrasensitive protein detection, the Biomarker Enumeration via Amplified Cavitation Optical Nanobubbles (BEACON) platform was developed. BEACON is a compartment-and enzyme-free high-throughput platform that enables digital counting of individual proteins. BEACON is a single-protein immunoassay that utilizes capture antibody-modified silica beads and detection antibody-conjugated AuNPs (FIG. 1A). Instead of enzymatic fluorescent amplification within physical microscale compartments, bead-protein-AuNP complexes are introduced into a fluidic channel, where individual AuNPs are excited by a pulsed laser. This excitation generates plasmonic nanobubbles (PNBs), which produce strong scattering signals within a laser-defined virtual compartment (FIG. 1B). The discrete PNB signals enable digital counting and absolute quantification of single protein molecules. Attomolar detection has been demonstrated for clinically relevant targets such as prostate-specific antigen (PSA, 32 aM), SARS-CoV-2 spike protein (COVID-S, 123 aM), and respiratory syncytial virus fusion protein (RSV-F, 65 aM). BEACON thus provides a single-molecule immunoassay platform that enables high-throughput, compartment-and enzyme-free counting of individual protein molecules.

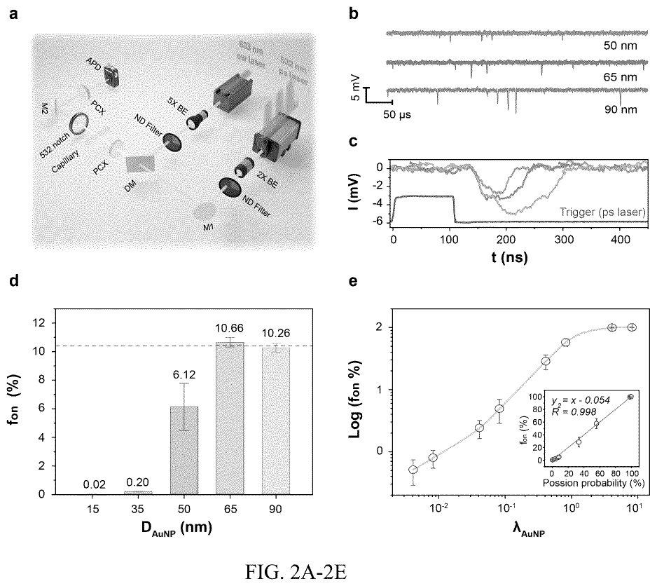

The capability of BEACON to detect single AuNPs was first evaluated, representing a critical step toward achieving single-protein counting. A compact and tunable configuration for nanobubble detection was implemented (FIG. 2A). A 532 nm picosecond pulsed laser was used to excite AuNPs and induce plasmonic nanobubble formation, while a co-aligned 633 nm continuous-wave laser enabled real-time scattering detection. Both beams were passed through beam expanders to match their diameters at the focal plane, and neutral density filters were employed to precisely control laser intensity. The beams were combined using a dichroic mirror and focused into a microcapillary through a plano-convex lens to form the virtual detection zone. Scattering signals were collected through a 532 nm notch filter and recorded by an amplified photodiode. To quantify the digital counting of individual nanoparticles, nanoparticle occupancy (average nanoparticle count per detection zone) was defined as λ, calculated based on known particle concentration and optical detection volume. The Poisson detection probability (1-e⁻) was compared with experimentally measured detection probabilities. High nanoparticle occupancy (λ_{AuNP}=100) showed consistent PNB generation for AuNPs≥35 nm at laser fluences above 0.3 J/cm². Single PNB characteristics were then systematically investigated for AuNPs of different sizes (50, 65, and 90 nm) at low nanoparticle occupancy (λ_{AuNP}=0.1) for single nanoparticle counting (FIGS. 2B-2C). Analysis revealed that both the amplitude and lifetime of individual nanobubble events increase with nanoparticle size, enhancing the detectability of larger AuNPs. Quantitative analysis was performed on AuNPs of different sizes at nanoparticle occupancy λ_{AuNP}=0.1 with a laser pulse energy of 1.7 J/cm² (FIG. 2D). For each particle size, three independent tests were conducted with 20,000 pulses each. Experimental results were compared with Poisson probability. At 1.7 J/cm², the system enables reliable counting of AuNPs that are at least 65 nm in diameter using PNBs and matches Poisson predictions closely. Counting of 65 nm AuNPs was then performed across different concentrations (FIG. 2E). The strong linear correlation between experimental fₒₙ and theoretical predictions (inset) supports the precision of the BEACON platform in single AuNP absolute quantification and its utility for subsequent single-protein counting.

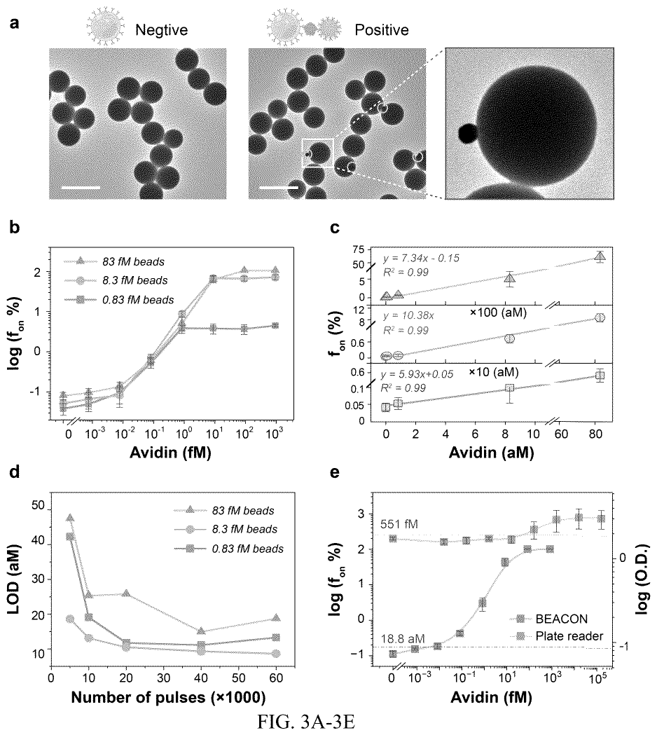

The BEACON assay was next evaluated using avidin as a model protein to characterize single-protein counting performance. 65 nm AuNPs were selected as the probe for subsequent experiments, as they reliably generate PNB signals that accurately count individual AuNPs. To form bead-avidin-AuNP sandwich complexes, biotin-functionalized silica beads (500 nm) were first incubated with avidin followed by a filtration step to remove unbound avidin. The beads were then incubated with biotinylated AuNPs to complete sandwich complex formation, followed by a second filtration washing step to eliminate excess AuNPs and residual reagents. Transmission electron microscopy (TEM) images confirmed successful complex formation, showing AuNPs localized on the bead surface only in the presence of avidin (FIG. 3A). PNB signal traces from 100 consecutive laser pulses demonstrated robust signal generation exclusively in avidin-positive samples (versus negative samples without avidin).

Having confirmed single-protein counting, assay parameters were examined to determine their influence on detection sensitivity and dynamic range. Specifically, three bead concentrations (0.83, 8.3, and 83 fM) were tested while keeping all other assay conditions constant. The calibration curves (FIG. 3B) show that the detection probability (fₒₙ) increases with protein concentration and eventually saturates when the number of protein molecules exceeds the number of available beads. At this point, nearly all beads are labeled, and the system can no longer distinguish further increases in protein concentration. FIG. 3C highlights the low-concentration regime, demonstrating that increasing bead concentration broadens the linear detection range. Strong linear correlations (R²=0.99) were observed across bead concentrations, indicating high signal fidelity and reproducibility. These results emphasize that optimizing the bead-to-protein ratio is critical for extending the linear dynamic range and maximizing assay performance at attomolar levels.

The role of counting statistics was then investigated by examining how varying the number of PNB detection pulses influences the limit of detection (LOD). Increasing the number of detection pulses initially lowers the LOD and, beyond a certain number of pulses (~40,000), the LOD improvement reaches a plateau (FIG. 3D). Calibration curves corresponding to different laser pulse counts showed that lower pulse numbers lead to greater variability, indicating reduced quantification accuracy. Additional pulses beyond the plateau do not significantly enhance sensitivity. Comparison of BEACON with colorimetric or analog detection of AuNPs showed that BEACON achieves a significantly lower LOD (18.8 aM) compared to colorimetric detection (551 fM, FIG. 3E). This highlights the advantage of PNB-based digital counting, which offers enhanced sensitivity and quantification accuracy over analog assays.

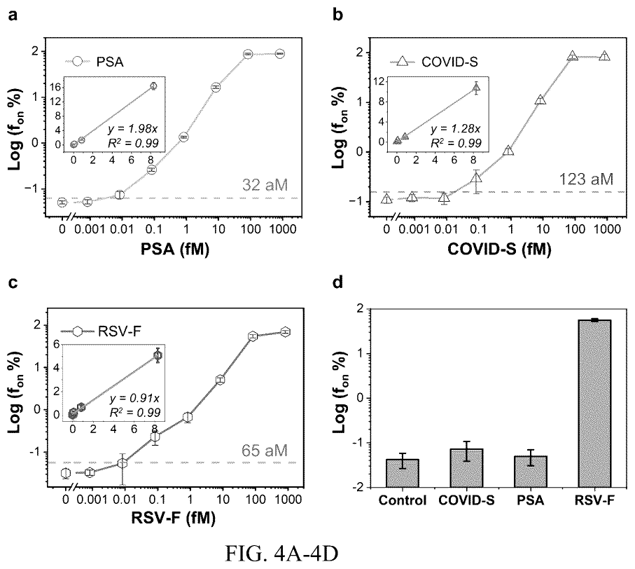

Having optimized key assay parameters, the practical performance of BEACON was evaluated under the established optimal conditions (83 fM bead concentration, 60,000 detection pulses). The platform was tested for detection of three clinically relevant protein biomarkers: prostate-specific antigen (PSA), SARS-CoV-2 spike protein (COVID-S), and respiratory syncytial virus fusion protein (RSV-F). PNB detection probability (fon) increased with target concentration, revealing a clear concentration-dependent response (FIGS. 4A-4C). Limits of detection were determined to be 32 aM for PSA, 123 aM for COVID-S, and 65 aM for RSV-F. Insets in each panel show linear correlation analyses in the 0-10 aM low-concentration regime with high linearity (R²=0.99) across all three biomarkers. These results establish that BEACON enables accurate, calibration-free digital quantification up to 100 fM. Compared with corresponding commercial ELISA kits, BEACON provides approximately four orders of magnitude improvement in sensitivity. Specificity was assessed through a cross-reactivity evaluation using the RSV-F detection configuration against non-target proteins, including COVID-S, PSA, and a blank control (no protein present). Each protein was tested at 160 fM under identical assay conditions, more than 1000-fold above the detection limit. The RSV-F sample exhibited significantly higher detection probability, while non-target proteins produced fon values at background levels (<0.1%), confirming high specificity of the BEACON assay (FIG. 4D).

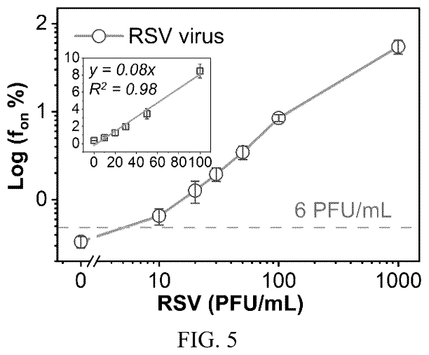

To further validate clinical utility for viral diagnostics, quantitative detection of respiratory syncytial virus was performed by targeting its surface fusion protein. RSV virus samples were serially diluted from 0 to 1000 PFU/mL, lysed using NP-40 buffer to disrupt the viral membrane and release surface proteins, and then detected using the BEACON protocol with RSV-F protein-specific antibody pairs. The limit of detection was determined to be approximately 6 PFU/mL (FIG. 5), corresponding to ~60 copies/mL based on prior calibration between plaque-forming units (PFU) and copy number using digital RNA quantification on the same samples (100 PFU corresponds to 1000 RNA copies). These results establish BEACON as a robust platform capable of detecting complex samples at the protein level, highlighting its potential for early-stage infection diagnosis.

The BEACON platform represents a significant advancement in digital protein detection, achieving attomolar sensitivity without enzymatic amplification or compartmentalization. BEACON also introduces several improvements over the original DIAMOND assay. First, it adopts the widely used dELISA immunoassay framework, enabling detection of diverse protein biomarkers, whereas DIAMOND targets only intact virus particles. Second, BEACON counts individual gold nanoparticles (AuNPs) instead of discriminating between aggregated and non-aggregated AuNP populations. Third, when evaluated with identical RSV samples, BEACON delivered a 16-fold sensitivity gain, achieving 6 PFU/mL (≈60 RNA copies/mL) compared with 100 PFU/mL (≈1000 RNA copies/mL). This improvement stems from two modifications: (1) a lysis buffer that disrupts the viral envelope and releases multiple surface fusion proteins, and (2) washing steps that reduce background noise and sharpen assay specificity. In contrast, the original DIAMOND protocol employs a homogeneous, wash-free format that prioritizes speed and simplicity at the cost of sensitivity.

Despite its advantages, BEACON has inherent limitations that require further development. One challenge lies in its multiplexing capacity: the binary on/off nature of plasmonic nanobubble signals enables highly sensitive digital counting but does not inherently support multiplexed detection based on signal intensity or lifetime. Additional labeling strategies would be needed to distinguish multiple analytes. One possible strategy involves incorporation of fluorescent bead-barcoding modalities to enable multiplexing by providing a direct measure of the unique fluorescent signal of each bead. Another limitation concerns the dynamic range and bead number control, which are intrinsically tied to Poisson statistics governing digital assays. Maintaining occupancy at 1 or 0 target per bead ensures digital counting accuracy. However, increasing the total bead number to achieve this condition proportionally extends the overall detection time, as each bead must be interrogated individually. This trade-off between statistical precision and throughput represents a common challenge among bead-based digital assays. In practical applications, predilution of concentrated samples or adjustment of bead numbers to match expected analyte abundance may be necessary to ensure accurate quantification while maintaining assay efficiency.

Readout throughput of BEACON could be further improved by employing high-repetition-rate lasers. Since each PNB event lasts only several hundred nanoseconds, the system theoretically supports up to 10⁶ readouts per second (MHz range) when paired with a high-frequency pulsed laser. Such rapid interrogation would substantially enhance counting throughput and enable real-time, high-density digital quantification of proteins, further extending BEACON's utility for large-scale clinical or multiplexed biomarker analysis.

In summary, BEACON offers a highly sensitive, rapid, and simplified digital protein counting method with significant potential for clinical diagnostics and biomarker discovery. Further developments in multiplexing, bead-to-protein ratio optimization, and high-repetition-rate laser pulses can position the BEACON platform as a scalable, high-throughput tool for next-generation bioanalytical applications.

### REFERENCES

1. Wu et al., Chem Soc Rev 2015, 44, 2963-97
2. Momenbeitollahi et al., Anal Bioanal Chem 2021, 413, 5995-6011
3. Barth et al., Light: Science & Applications 2025, 14, 177
4. Usha et al., Chem Soc Rev 2021, 50, 13012-13089
5. Kelley, ACS Sens 2017, 2, 193-197
6. O'Connell et al., Neurol Res 2020, 42, 346-353
7. Hosseini et al., Enzyme-linked Immunosorbent Assay (ELISA), 2018, 67-115
8. Peng et al., Trac-Trends in Analytical Chemistry 2022, 152, 116605
9. Kuo et al., Acc Chem Res 2023, 56, 1031-1042
10. Duffy, Lab Chip 2023, 23, 818-847
11. Ven et al., ACS Sens 2018, 3, 264-284
12. Wu et al., Adv Mater 2020, 32, e1904339
13. Gooding et al., Angew. Chem 2016, 55, 11354-66
14. Rissin et al., Nat Biotechnol 2010, 28, 595-9
15. Perez-Ruiz et al., Anal Chim Acta 2018, 1015, 74-81
16. Stephens et al., Biosens Bioelectron 2023, 237, 115536
17. Cohen et al., ACS Nano 2020, 14, 9491-9501
18. Yelleswarapu et al., Proc Natl Acad Sci U S A 2019, 116, 4489-4495
19. Yi et al., Sensors and Actuators B-Chemical 2022, 369, 132214
20. Jing et al., ACS Nano 2019, 13, 8609-8617
21. Monroe et al., Analytical Chemistry 2013, 85, 3698-3706
22. Zhang et al., Nanoscale Advances 2025, 7, 5720-5734
23. Zhou et al., Light: Science & Applications 2025, 14, 76
24. Wu et al., J Am Chem Soc 2020, 142, 12314-12323
25. Wu et al., ACS Nano 2022, 16, 1025-1035
26. Zhang et al., ACS Nano 2024, 18, 29891-29901
27. Lukianova-Hleb et al., ACS Nano 2010, 4, 2109-23
28. Moon et al., Journal of Physical Chemistry C 2021, 125, 25357-25368
29. Spellauge et al., Light: Science & Applications 2022, 11, 68
30. Liu et al., Nat Commun 2022, 13, 1687
31. Parsamian et al., ACS Nano 2023, 17, 7797-7805
32. Basu, SLAS Technol 2017, 22, 369-386
33. Rissin et al., Nano Lett 2006, 6, 520-3
34. Shin et al., Small 2019, 15, e1804476

1. A method for digital detection of a target analyte in a sample without enzymatic amplification or physical compartmentalization, comprising: (a) capturing the target analyte on a solid support using a binding agent forming a captured target analyte; (b) labeling the captured target analyte with a plasmonic nanoparticle forming a labeled solid support; (c) exposing the labeled solid support to a pulsed laser light generating plasmonic nanobubbles around the plasmonic nanoparticles; (d) detecting scattering signals from the plasmonic nanobubbles; and (e) digitally quantifying the target analyte based on the detected scattering signals.

2. The method of claim 1, wherein the target analyte is a protein biomarker.

3. The method of claim 1, wherein the solid support is a microbead.

4. The method of claim 3, wherein the microbead is a silica microbead or magnetic microbead.

5. The method of claim 4, wherein the silica microbead has a diameter of about 0.5 to 5 micrometers.

6. The method of claim 1, wherein the plasmonic nanoparticle is selected from the group consisting of a gold nanoparticle, a silver nanoparticle, a copper nanoparticle, an alloy thereof, and combinations thereof, and wherein the plasmonic nanoparticle has a shape selected from the group consisting of spherical, rod-shaped, shell-shaped, and star-shaped.

7. The method of claim 1, wherein the plasmonic nanoparticle is a gold nanoparticle.

8. The method of claim 7, wherein the gold nanoparticle has a diameter of at least 55 nm.

9. The method of claim 8, wherein the gold nanoparticle has a diameter of about 65 nm.

10. The method of claim 1, wherein the binding agent is selected from the group consisting of an antibody, an antibody fragment, an aptamer, a peptide, a lectin, a receptor, a ligand, a nucleic acid probe, an affibody, a nanobody, and combinations thereof.

11. The method of claim 1, wherein the binding agent is an antibody.

12. The method of claim 11, wherein capturing and labeling form a sandwich immunocomplex.

13. The method of claim 1, further comprising removing unbound plasmonic nanoparticles after labeling and before exposing to pulsed laser light.

14. The method of claim 12, wherein removing comprises washing by filtration or centrifugation or magnetic separation.

15. The method of claim 1, wherein the labeled solid supports are flowed through a detection zone during exposure to pulsed laser light and detection.

16. The method of claim 15, wherein the detection zone defines a virtual detection volume of about 80 pL formed by overlapping beams of the pulsed laser light and a continuous-wave probe laser.

17. The method of claim 15, wherein the detection zone is in a fluidic channel or microcapillary.

18. The method of claim 1, wherein the pulsed laser light has a pulse duration of less than about 100 nanoseconds and a fluence of about 0.3 J/cm² to about 2 J/cm².

19. The method of claim 1, wherein the pulsed laser light has a wavelength of about 532 nm.

20. The method of claim 1, wherein digitally quantifying comprises counting binary (on/off) scattering signals and applying Poisson statistics to determine absolute concentration.

21. The method of claim 1, wherein the method achieves attomolar sensitivity.

22. The method of claim 2, wherein the protein biomarker is selected from the group consisting of prostate-specific antigen (PSA), SARS-CoV-2 spike protein, and respiratory syncytial virus fusion protein (RSV-F).

23. The method of claim 1, performed at room temperature.

24. The method of claim 1, wherein the sample volume is less than 10 μL.

25. A method for digital detection of a multivalent target analyte in a sample without enzymatic amplification or physical compartmentalization, comprising: (a) mixing the sample with a plurality of pairs of plasmonic nanoparticles, wherein each member of a pair is conjugated to a different binding agent that recognizes a different epitope on the multivalent target analyte, and wherein binding of the multivalent target analyte to both binding agents of a pair induces aggregation of the plasmonic nanoparticles; (b) exposing the mixture to pulsed laser light sufficient to generate plasmonic nanobubbles preferentially around aggregated plasmonic nanoparticles compared to non-aggregated plasmonic nanoparticles; (c) detecting transient scattering signals from the plasmonic nanobubbles using a continuous-wave probe laser and a photodetector; and (d) digitally quantifying the target analyte by counting scattering events that satisfy a threshold criterion indicative of aggregation, wherein the method distinguishes aggregated from non-aggregated plasmonic nanoparticles based on at least one of: scattering signal amplitude, scattering signal duration, nanobubble generation threshold fluence, or nanobubble lifetime.

26. The method of claim 25, wherein the plasmonic nanoparticles are gold nanoparticles having a diameter of about 40 nm to about 70 nm.

27. The method of claim 25, wherein the gold nanoparticles have a diameter of about 50 nm to about 65 nm.

28. The method of claim 25, wherein the multivalent target analyte is an intact virus particle, a viral capsid, a protein complex, or a multimeric protein.

29. The method of claim 28, wherein the intact virus particle is a respiratory syncytial virus (RSV) particle.

30. The method of claim 25, wherein the method is performed as a wash-free, homogeneous assay.

31. The method of claim 25, wherein the mixture is flowed through a microfluidic channel or microcapillary during exposure to the pulsed laser light and detection of scattering signals.

32. The method of claim 25, wherein the pulsed laser light has a wavelength of about 520 nm to about 550 nm and a pulse duration of less than about 10 nanoseconds.

33. The method of claim 25, wherein the continuous-wave probe laser has a wavelength of about 600 nm to about 650 nm.

34. The method of claim 25, wherein the threshold criterion for counting a scattering event as indicative of aggregation comprises at least one of: (a) a scattering signal amplitude at least 2-fold greater than the mean amplitude of signals from non-aggregated nanoparticles; (b) a nanobubble lifetime at least 1.5-fold longer than the mean lifetime from non-aggregated nanoparticles; or (c) a nanobubble generation fluence threshold at least 20% lower than the threshold for non-aggregated nanoparticles.

35. The method of claim 25, wherein the digital quantification is performed using Poisson statistics applied to the fraction of detected scattering events that satisfy the aggregation threshold criterion.

36. The method of claim 25, wherein the method achieves detection of the multivalent target analyte at a concentration of 100 PFU/mL or lower.

37. The method of claim 36, wherein the method achieves detection of intact respiratory syncytial virus at a concentration of about 100 PFU/mL or lower.

38. The method of claim 25, wherein the entire assay is completed in less than 30 minutes from sample addition to final quantification.

39. The method of claim 25, wherein the sample volume used is less than 10 μL.

40. A system for digital detection of a target analyte without enzymatic amplification or physical compartmentalization, comprising: (a) solid supports configured to capture the target analyte; (b) plasmonic nanoparticles configured to label the captured target analyte; (c) a pulsed laser configured to generate plasmonic nanobubbles around the plasmonic nanoparticles; (d) a detector configured to detect scattering signals from the plasmonic nanobubbles; and (e) a processor configured to digitally quantify the target analyte based on the scattering signals.

41. The system of claim 40, further comprising a continuous-wave probe laser co-aligned with the pulsed laser, and wherein the detector includes a spectral filter and an amplified photodetector configured for microsecond-resolution detection.

42. The system of claim 40, wherein the solid supports are silica microbeads coated with capture antibodies, and the plasmonic nanoparticles are gold nanoparticles conjugated to detection antibodies.

43. The system of claim 40, further comprising a fluidic channel for flowing labeled supports through a laser interrogation zone.

44. The system of claim 40, wherein the detector is a photodetector, and further comprising a probe laser for illuminating the nanobubbles to enhance scattering detection.

45. The system of claim 40, wherein the processor is configured to apply Poisson statistics for absolute quantification.

46. A system for digital detection of a target analyte without enzymatic amplification or physical compartmentalization, comprising: (a) antibody-conjugated plasmonic nanoparticles configured to aggregate upon binding to the target analyte; (b) a pulsed laser configured to generate plasmonic nanobubbles around the aggregated plasmonic nanoparticles; (c) a detector configured to detect scattering signals from the plasmonic nanobubbles; and (d) a processor configured to digitally quantify the target analyte based on the scattering signals distinguishing aggregated from non-aggregated plasmonic nanoparticles.

47. A kit for digital detection of a target analyte, comprising: (a) solid supports functionalized with binding agents for capturing the target analyte; (b) plasmonic nanoparticles functionalized with binding agents for labeling the captured target analyte; and (c) instructions for exposing labeled supports to pulsed laser light to generate and detect plasmonic nanobubbles for digital quantification without enzymatic amplification or physical compartmentalization.

48. The kit of claim 47, wherein the solid supports are silica microbeads, and the plasmonic nanoparticles are gold nanoparticles.

49. The kit of claim 47, further comprising buffers for incubation and washing.

50. A kit for digital detection of a target analyte, comprising: (a) pairs of antibody-conjugated plasmonic nanoparticles configured to aggregate upon binding to the target analyte; and (b) instructions for exposing a mixture of the plasmonic nanoparticles and sample to pulsed laser light to generate and detect plasmonic nanobubbles for digital quantification.

51. A method for digital detection of a viral protein in a lysed virus sample without enzymatic amplification or physical compartmentalization, comprising: (a) lysing a virus sample to release a viral protein; (b) capturing the viral protein on a solid support using a binding agent forming a captured viral protein; (c) labeling the captured viral protein with a plasmonic nanoparticle forming a labeled solid support; (d) exposing the labeled solid support to a pulsed laser light generating plasmonic nanobubbles around the plasmonic nanoparticles; (e) detecting scattering signals from the plasmonic nanobubbles; and (f) digitally quantifying the viral protein based on the detected scattering signals, wherein the method achieves detection at a concentration of about 6 PFU/mL or lower.
